(19) United States
(12) Patent Application Publication
(10) Pub. No.: US 2013/0337476 A1
(43) Pub. Date: Dec. 19, 2013

(54) URINARY sCD14 AS A BIOMARKER FOR CORONARY ARTERY DISEASE

(71) Applicants: Min-Yi Lee, Kaohsiung City (TW); Shyh-Horng Chiou, Kaohsiung City (TW); Wen-Jen Chen, Taitung County (TW)

(72) Inventors: Min-Yi Lee, Kaohsiung City (TW); Shyh-Horng Chiou, Kaohsiung City (TW); Wen-Jen Chen, Taitung County (TW)

(21) Appl. No.: 13/495,442

(22) Filed: Jun. 13, 2012

(51) Int. Cl. G01N 33/566; G01N 21/64; G01N 21/76

(52) U.S. Cl. 435/7.92; 436/501; 435/7.9

(57) ABSTRACT

Disclosed herein is a method for the detection or preliminary screening of coronary artery disease, including: obtaining a urine sample from a human subject suspected of having coronary artery disease; detecting a level of sCD14 in the urine sample from the human subject suspected of having coronary artery disease; and comparing the detected level of sCD14 in the urine sample with a predetermined standard, wherein the level of sCD14 in the urine sample from the human subject suspected of having coronary artery disease greater than the predetermined standard is indicative of the presence of coronary artery disease.

### BACKGROUND OF THE INVENTION

1. Field of the Invention

This invention relates to use of soluble CD14 (sCD14) from a urine sample of a human subject as a biomarker for human coronary artery disease. Particularly, this invention provides a method for the detection, preliminary screening or monitoring of coronary artery disease, in which the human subject is determined to have the coronary artery disease if the level of sCD14 in the urine sample of the human subject is higher than that of a standard.

2. Description of the Related Art

Coronary artery disease (CAD) is the leading cause of death worldwide. It is also known as coronary heart disease (CHD) and atherosclerotic heart disease. CAD is caused by stenosis and/or obstruction of the vessels. Symptoms of CAD patients include: myocardial ischemia, myocardial infarction, angina pectoris, ischemic cardiomyopathy, congestive heart failure and aneurysm formation. CAD patients are usually identified after the onset of symptoms. Thus, much research has been devoted to the development of a reliable biomarker for detecting and monitoring CAD.

It has been disclosed that several molecules either in the serum or plasma are strongly associated with the presence of CAD and can act as a biomarker to predict the risk for CAD. It was disclosed by Greco T. P. et al that oxidized low-density lipoprotein/beta(2)-glycoprotein I complexes are associated with the severity of CAD (Greco T. P. et al. (2010), Am. J. Clin. Pathol., 133:737-743). Inoue T. et al. disclosed that lipocalin-type prostaglandin D synthase is positively correlated with the severity of stable coronary artery disease (Inoue T. et al. (2008), Atherosclerosis, 201:385-391). In addition, it was disclosed by Harsimran K. et al. that monocyte chemoattractant protein-1 can potentially serve as a biomarker for CAD (Harsimran K. et al. (2009), Diab. Vasc. Dis. Res., 6:288-290). Moreover, Niccoli G. et al. disclosed that eosinophil cationic protein is a biomarker for CAD (Niccoli G. et al. (2010), Atherosclerosis, 211:606-611).

Certain proteins in urine are associated with the presence of CAD. It was disclosed by Fitzsimmons P. J. et al. that elevated levels of matrix metalloproteinases-9 (MMP-9) and tissue inhibitor of metalloproteinases-1(TIMP-1) have been observed in patients with CAD or acute coronary syndrome (P. J. Fitzsimmons et al. (2007), Atherosclerosis, 194:196-203). It was also disclosed by Basarici I. et al. that 8-isoprostane was significantly increased in patients with CAD (I. Basarici et al. (2008), Acta. Cardiol., 63:415-422).

CD16 is a Fc receptor that can bind to the Fc portion of IgG. It is found on the surfaces of natural killer cells, monocytes and macrophages.

CD14 is a glycoprotein associated with innate immune system, and acts as a co-receptor for the detection of bacteriallipopolysaccharides (LPS). CD14 exists in two forms in a human body:

(1) Membrane Bound CD14 (mCD14):
mCD14 is a 55 kDa glycosylphosphatidylinositol (GPI)-linked protein. mCD14 is anchored through the glycosylphosphatidylinositol (GPI) tail to a surface membrane of myeloid cells, such as monocytes, macrophages, neutrophils and polymorphonuclear phagocytes.

(2) Soluble CD14 (sCD14):
sCD14 has the same amino acid sequence with that of mCD14, except it lacks the GPI tail. sCD14 is formed in hepatocytes and monocytes and secreted outside the hepatocytes and monocytes before coupling to the GPI tail (approximate molecular weight is 56 kDa). The digestion of mCD14 by phospholipase or protease allows the shedding of GPI tail from mCD14 so as to form sCD14 (approximate molecular weight is 48 kDa). Thus sCD14 primarily exists in plasma or serum.

CD14⁺CD16⁺ monocytes are proinflammatory monocytes, which have increased capacity to secrete proinflammatory cytokines, e.g., tumor necrosis factor-alpha (TNF-α). It was disclosed by Schlitt A. et al. that increased level of CD14⁺CD16⁺ monocytes is associated with CAD and elevated levels of TNF-α. Thus, CD14⁺CD16⁺ monocytes suggest a possibly important role in the development of atherosclerosis (A. Schlitt et al. (2004), Thromb. Haemost., 92:419-24).

WO 2011/083145 A1 discloses that the level of CD14 in exosome can be used as a biomarker for the prognosis of risk on cardiovascular events, such as stroke, transient ischemic attack, myocardial infarction and cerebral bleeding. Specifically, CD14 from plasma exosomes used in conjunction with traditional risk factors (e.g., gender, age, cholesterol, systolic blood pressure etc.) for CAD showed an increase in the area under the ROC curve (AUC) than the traditional risk factors alone (AUC for CD14 and the traditional risk factors vs. AUC for the risk factors alone are 0.778 vs. 0.630). The results suggest an increased discriminative power of CD14 from the exosome for the prognosis of future cardiovascular events.

Studies have disclosed increased level of sCD14 from plasma or serum of a subject with inflammatory disease, such as sepsis, rheumatoid arthritis (G. Horneff et al. (1993), Clin. Exp. Immunol., 91:207-213), systemic lupus erythematosus (W. A. Nockher et al. (1994), Clin. Exp. Immunol., 96:15-19) and Kawasaki disease (S. Takeshita et al. (2000), Clin. Exp. Immunol., 119:376-381).

R. G. Tang et al. and J. W. Zhu et al. disclosed the level of sCD14 in the serum is positively correlated with the severity of CAD, thus suggesting the potential of using sCD14 in the serum as a biomarker for CAD (R. G. TANG et al. (2007), Chin J. Crit. Care Med., 27:326-328; J. W. ZHU and C. Y. LIU (2008), Acta Academiae Medical Qingdao Universitatis, 44:156-157, 161).

While plasma and serum collection from the blood is invasive, non-invasive approaches to collect specimen are considered more convenient and a greater quantity can be collected at a lower cost when compared to the invasive method. Thus, the object of the present invention is to provide a biomarker, specifically sCD14, from the urine sample, for the detection and monitoring of CAD accompanied by high specificity and sensitivity.

### SUMMARY OF THE INVENTION

According to a first aspect, the present invention provides a method for the detection or preliminary screening of coronary artery disease, which comprises:
obtaining a urine sample from a human subject suspected of having coronary artery disease;
detecting a level of sCD14 in the urine sample from the human subject suspected of having coronary artery disease; and
comparing the detected level of sCD14 in the urine sample with a predetermined standard;
wherein the level of sCD14 in the urine sample from the human subject suspected of having coronary artery disease greater than the predetermined standard is indicative of the presence of coronary artery disease.

According to a second aspect, the present invention also provides a method for the monitoring of coronary artery disease, comprising:
periodically obtaining a urine sample from a human subject suspected of having coronary artery disease;
detecting a level of sCD14 in the urine sample from the human subject suspected of having coronary artery disease; and
comparing the detected level of sCD14 in the urine sample with a predetermined standard;
wherein the level of sCD14 in the urine sample from the human subject suspected of having coronary artery disease greater than the predetermined standard is indicative of the presence of coronary artery disease.

### BRIEF DESCRIPTION OF THE DRAWINGS

Other features and advantages of the present invention will become apparent in the following detailed description of the preferred embodiments of the invention, with reference to the accompanying drawings, in which:

FIG. 1 is a plot showing the relative expression of urinary soluble CD14 (sCD14) from healthy subjects and CAD patients with different SYNTAX scores, the relative expression of urinary sCD14 being derived from analysis of band intensity of a western blot using scanning densitometry software (Multi Gauge V3.0); and

FIG. 2 is a receiver-operating characteristic (ROC) plot of urinary sCD14 for the diagnosis of coronary artery disease. Using a standard of 3.51 μg/ml for urinary sCD14, the prediction of coronary artery disease has a sensitivity of 0.838 and a specificity of 0.703. Area under the ROC curve (AUC) is 0.846. The dashed-line curve is a reference curve having an AUC=0.5.

### DETAILED DESCRIPTION OF THE INVENTION

It is to be understood that, if any prior art publication is referred to herein, such reference does not constitute an admission that the publication forms a part of the common general knowledge in the art, in Taiwan or any other country.

For the purpose of this specification, it will be clearly understood that the word “comprising” means “including but not limited to”, and that the word “comprises” has a corresponding meaning.

Unless otherwise defined, all technical and scientific terms used herein have the meaning commonly understood by a person skilled in the art to which this invention belongs. One skilled in the art will recognize many methods and materials similar or equivalent to those described herein, which could be used in the practice of this invention. Indeed, this invention is in no way limited to the methods and materials described. For clarity, the following definitions are used herein.

The terms “coronary artery disease (CAD)”, “coronary heart disease (CHD)” and “atherosclerotic heart disease” are used interchangeably in the present specification, and are defined as a disease with occluded coronary arteries due to the buildup of cholesterol and plaque on the inner walls of the artery. Clinical signs and symptoms include, but are not limited to, the following: myocardial ischemia, myocardial infarction, angina pectoris, ischemic cardiomyopathy, congestive heart failure and aneurysm formation.

The present invention provides a method for the monitoring, detection or preliminary screening of coronary artery disease, which comprises:
obtaining a urine sample from a human subject suspected of having coronary artery disease;
detecting a level of sCD14 in the urine sample from the human subject suspected of having coronary artery disease; and
comparing the detected level of sCD14 in the urine sample with a predetermined standard;

wherein the level of sCD14 in the urine sample from the human subject suspected of having coronary artery disease greater than the predetermined standard is indicative of the presence of coronary artery disease.

In the present invention, the urine sample can be obtained anytime from the human subject. Preferably, the urine sample is the first urinated sample in the morning, more preferably, the midstream of the first urinated sample in the morning.

According to the present invention, the level of sCD14 can be determined by any means that is known to those skilled in the art. Preferably, the level of sCD14 can be determined by immunoassays. Examples of the immunoassay include multiplex immunoassay, enzyme linked immunosorbent assay (ELISA), radioimmunoassay (RIA), immunoradiometric assay (IRMA), fluorescent immunoassay (FIA), chemiluminescent immunoassay and immunonephelometry.

The level of sCD14 is determined using an antibody-based binding moiety which specifically binds to sCD14. In an example of this invention, the antibody-based binding moiety is an antibody.

“Antibody-based binding moiety” or “antibody” includes immunoglobulin molecules and immunologically active determinants of immunoglobulin molecules, e.g., molecules that contain an antigen-binding site that specifically binds to sCD14. The term “antibody-based binding moiety” is intended to include whole antibodies of any isotype (e.g., IgG, IgA, IgM, IgE, etc.), and fragments thereof that are also specifically reactive with sCD14.

In this invention, the antibody-based binding moiety includes polyclonal, monoclonal or other purified preparations of antibodies and recombinant antibodies, and is further intended to include humanized antibodies, bi-specific antibodies, and chimeric molecules having at least one antigen-binding determinant derived from an antibody molecule.

In this invention, “antibody-based binding moiety” or “antibody” includes a capture antibody and a detecting antibody.

The term “capture antibody” as used herein is defined as an antibody, whether monoclonal, polyclonal or of an immunoreactive fragment, which is capable of binding to an antigen of interest, and thereby allows the recognition of the antigen by a subsequently applied antibody. The capture antibody can be used in either a heterogeneous (solid phase) or homogeneous (solution phase) assay. Preferably, the capture antibody is immobilized onto a solid phase.

The term “detecting antibody” as used herein is defined as an antibody having a detectable label that is specific for (i.e., binds, is bound by, or forms a complex with) one or more analytes of interest in a sample. The term also encompasses an antibody that is specific for one or more analytes of interest, wherein the antibody can be bound by another species that comprises a detectable label. Examples of detectable labels include, but are not limited to, a radioactive label, a hapten label, a fluorescent label, a chemiluminescent label, an enzymatic label, a nucleotide (e.g., oligonucleotide) label, an epitope tag, and combinations thereof.

Examples of hapten label include biotin/streptavidin and digoxigenin.

Examples of epitope tag include T7, c-Myc, HA, VSV-G, HSV, FLAG, V5 and HIS.

Antibodies can be fragmented using conventional techniques. The term “fragment(s) thereof” refers to segments of proteolytically-cleaved or recombinantly-prepared portions of an antibody molecule that are capable of selectively reacting with a certain protein.

Non-limiting examples of proteolytically-cleaved fragments and/or recombinantly-prepared portions include Fab, F(ab′)2, Fab′, Fv, dabs and a single-chain variable fragment (scFv) containing a VL and VH domain joined by a peptide linker. The scFv's may be covalently or non-covalently linked to form antibodies having two or more binding sites.

According to the present invention, the level of sCD14 is positively correlated to the intensity of the signal emitted from the detectably labeled antibody.

In one preferred embodiment of the present invention, the antibody-based binding moiety is detectably labeled by linking the antibody to an enzyme. The enzyme, in turn, when exposed to its substrate, will react with a substrate in such a manner as to produce a chemical moiety which can be detected, for example, by spectrophotometric, fluorometric or visual means. The enzymes which can be used to react with the detactable label of the antibodies of the present invention include, but are not limited to, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, β-galactosidase, ribonuclease, urease, catalase, glucose-VI-phosphate dehydrogenase, glucoamylase and acetylcholinesterase.

An antibody can also be labeled with a fluorescent compound. When the fluorescently labeled antibody is exposed to light of the proper wavelength, its presence can be detected due to fluorescence. Fluorescent compounds suitable for the present invention include, but are not limited to, CYE dyes, fluorescein isothiocyanate (FITC), rhodamine, phycoerythrin, coriphosphine-O (CPO), phycocyanin (PE), allophycocyanin (APC), o-phthaldehyde, fluorescamine and tandem dyes.

Examples of tandem dyes include PE-Cy5 (PC5), PE-Cy7 (PC7) and PE-Texas Red.

The antibody can be detected by fluorescence emitting metals, such as ¹⁵²Eu or other lanthanide series. These metals can be attached to the antibody by means of a metal-chelating groups such as diethylenetriaminepentaacetic acid (DTPA) or ethylenediaminetetraacetic acid (EDTA).

The antibody can be detected by coupling it to a chemiluminescent. The presence of the chemiluminescent-antibody is then determined by detecting the presence of luminescence that arises during the course of a chemical reaction. Examples of chemiluminescent compounds include, but are not limited to, luminol, luciferin, isoluminol, theromatic acridinium ester, imidazole, acridinium salt and oxalate ester.

Detection may also be accomplished using any of a variety of other immunoassays. For example, by radioactively labeling an antibody, it is possible to detect the antibody through the use of radioimmune assays. Means of detecting radioactive isotopes include, but are not limited to, gamma counter, scintillation counter and autoradiography. The antibody can be detected by radioactive isotope. Examples of radioactive isotope include, but are not limited to, ³H, ³¹P, ³⁵S, ¹⁴C, and ¹²⁵I.

According to the present invention, the term “predetermined standard” may indicate a range, a value or a cutoff value of the level of sCD14 from a urine sample of a healthy individual, which is determined by selected means. The cutoff value between a population of healthy individuals and subjects that have CAD can be determined by those skilled in the art.

The terms “healthy individual” and “normal subjects” can be used interchangeably to mean an individual not having any symptoms associated with CAD or not at risk of developing CAD, i.e. an individual considered healthy after evaluation by a professional medical practitioner.

Healthy individuals have normal coronary artery, and do not have a history of active infectious disease, prior stroke, acute coronary syndrome and malignancies.

In order to assess the progression of CAD or the effectiveness of a treatment for CAD, the level of urinary sCD14 as detected in a previous examination may also be used as a reference for the human subject.

In a preferred embodiment of the present invention, the predetermined standard for urinary sCD14 is determined by human sCD14 enzyme linked immunosorbent assay kit (Cat. No. HK320, Hycult Biotechnology, Uden, the Netherlands), giving a standard value of 3.51 μg/mL as the cutoff value of sCD14 to determine the presence of CAD.

### EXAMPLES

The present invention will be described in more detail with reference to the following examples, which are given for the purpose of illustration only and are not intended to limit the scope of the present invention.

### <Subjects>:

108 human subjects (69 men and 39 women) were recruited in the study of this invention using a protocol approved by the Medical Ethics Committee of Kaohsiung Municipal United Hospital. Written informed consents were obtained from all human subjects. Exclusion criteria were applied to all of the studied subjects, which included a history of active infectious disease, prior stroke, acute coronary syndrome and malignancies.

73 human subjects from cardiovascular internal medicine were proven to have coronary artery disease (CAD) by angiography, are hereinafter referred to as a CAD group. 35 human subjects had normal coronary artery, and are hereinafter referred to as a control group. The clinical characteristics, such as sex, age, SYNTAX score, and number of diseased vessel, are shown in Table 1. SYNTAX score was determined according to Sianos G. et al. (2005), Eurontervention, 1:219-227, and is herein incorporated by reference. The SYNTAX score was established on the basis of the characterization of coronary vasculature with respect to the severity of coronary artery disease and complexity of the lesion. Higher SYNTAX scores were hypothesized to represent more complex and severe CAD.

TABLE 1

| | | Control | CAD group |
|---|---|---|---|
| Number of studies subjects | | 35 | 73 |
| Age^{a} (range) | | 61.1 ± 13.4 | 66.5 ± 11.7 |
| | | (41~72) | (46~82) |
| sex(female/male) | | 14/21 | 31/42 |
| SYNTAX score^{a} | | 0 | 28 ± 14 |
| Number of | single-vessel | NA^{b} | 20 |
| diseased | disease(number of | | |
| vessel | subjects) | | |
| | multi-vessel | NA | 53 |
| | disease(number of | | |
| | subjects) | | |

^{a}shown as mean ± standard deviation
^{b}not applicable
^{c}: multi-vessel disease indicates 2 or 3 diseased vessels

<Sample Collection>

1. Serum Samples
Blood was collected after at least 8 hours of overnight fasting from radial artery. Serum was collected by centrifuging blood at 3,000 rpm for 10 minutes at 4° C.

2. Urine Samples
A midstream of a first morning urine specimen was collected in a sterile container from every human subject. The urine specimen was subsequently centrifuged at 1,000 g for 5 minutes to further remove cellular debris.

<Statistical Anaylsis>
Baseline characteristics were calculated for the CAD and control groups. Numeric data are presented as means±standard deviation (SD). The ANOVA and Chi-square tests were used for comparisons among the groups. A probability value less than 0.05 (p<0.05) was considered to indicate statistical significance.

### Example 1

### Analysis of the Level of Urinary sCD14 Between the Control and CAD Groups

To assess the association between the severity of CAD and the level of urinary sCD14, urine samples from the control group and CAD group with patients having different SYNTAX scores were obtained to determine the level of sCD14.

A. Preparation of Protein Samples from Urine

Urine samples for the following western blot analysis were obtained from two healthy individuals in the control group with SNYTAX score of 0, and 6 patients in the CAD group with SYNTAX scores of 2, 2, 4, 12, 25 and 34, respectively, torepresent different complexities and severities of CAD.

7 mL of the urine samples from these 8 subjects were collected according to the aforesaid section <Sample collection>, 2. Urine samples. 7 mL of 10% trichloroacetic acid (TCA) containing 6 mM dithiothreitol (DTT) was added into each of the urine samples, followed by reaction on ice for 30 minutes and centrifugation at 13,000 rpm for 30 minutes at 4° C. to obtain a protein precipitate. The protein precipitate was washed twice with ice-cold 100% acetone/6 mM DTT, followed by centrifugation at 13,000 rpm. The precipitate was then suspended in 0.3 ml of rehydration buffer (9.8 M urea, 0.5% Triton-100, 65 mM DTT and 0.5% ampholytes), thus obtaining a protein sample for further analyses.

The protein concentration was determined using 2-D Quant Kit (Amersham Biosciences, Piscataway, N.J.).

### B. Western Blot Analysis

Sodium dodecyl sulfate-polyacrylamide gel electrophoresis (SDS-PAGE) was used for western blot analysis. 10 μg of the protein sample obtained from the aforesaid section A was used. 5-fold amounts of a sample loading buffer (10% SDS, 0.3125 M Tris-HCl (pH 6.8), 10% glycerol, 0.5M DTT, and 0.01% bromphenol blue) were added to the protein sample. Protein denaturation was performed at 95° C. for 5 minutes.

Gel electrophoresis was used to analyze the proteins by using a SDS-polyacrylamide gel (12.5% separating gel and 5% stacking gel) in a gel electrophoresis apparatus (Hoefer SE260, Amersham Biosciences, Buckinghamshire, UK). A voltage of 80V was applied for 30 minutes followed by a voltage of 100V for the remaining course of electrophoresis. Proteins were allowed to migrate until bromophenol blue dye reached the bottom of the gel. The electrophoresis was performed at 8° C.

The proteins in the SDS-polyacrylamide gel were transferred to a poly vinylidene difluoride (PVDF) membrane (Millipore, Bed-ford, MA, USA) under 40 mA for 12 hours using a TE 22 Mini Tank Transfer Unit (Amersham Biosciences, Buckinghamshire, UK). The membrane was subjected to a blocking step in Tris-buffered saline with 0.1% Tween-20 (TBS-T) containing 5% nonfat dry milk for one hour and washed with TBS-T three times (10 minutes for each time), followed by incubation with rabbit anti-CD 14 polyclonal antibody (catalog number: GTX101342, Genetex, San Antonio, Tex., USA, a primary antobody) at 1:1000 in TBS-T overnight at 4° C. Thereafter, the membrane was washed three times with TBS-T followed by incubation with a secondary antibody, goat anti-rabbit IgG conjugated with horseradish peroxidase (diluted 1:500 in TBS-T, catalog no. L3032, Signalway Antibody, Pearland, Tex., USA). After 1 hour incubation at room temperature, the membrane was washed three times with TBS-T, 10 minutes for each time. Thereafter, the membrane was incubated with enhanced chemiluminescence substrate (ECL kit; Pierce, Rockford, Ill., USA). A ChemiDoc™ XRS⁺ System (Bio-Rad, Hercules, Calif., USA) was used to capture images of the membrane. A scanning densitometry software (Multi Gauge V3.0) was used for densitometric analysis to evalute signal intensity.

A blank area was used as a background to quantify the relative expression of sCD14.

The relative expression of sCD14 can be quantified by formula (I):

A=(B−C)/C×100% (I)
wherein,
   A=the relative expression of sCD14 (%)
   B=the signal intensity of sCD14
   C=the signal intensity of the background

### Results:

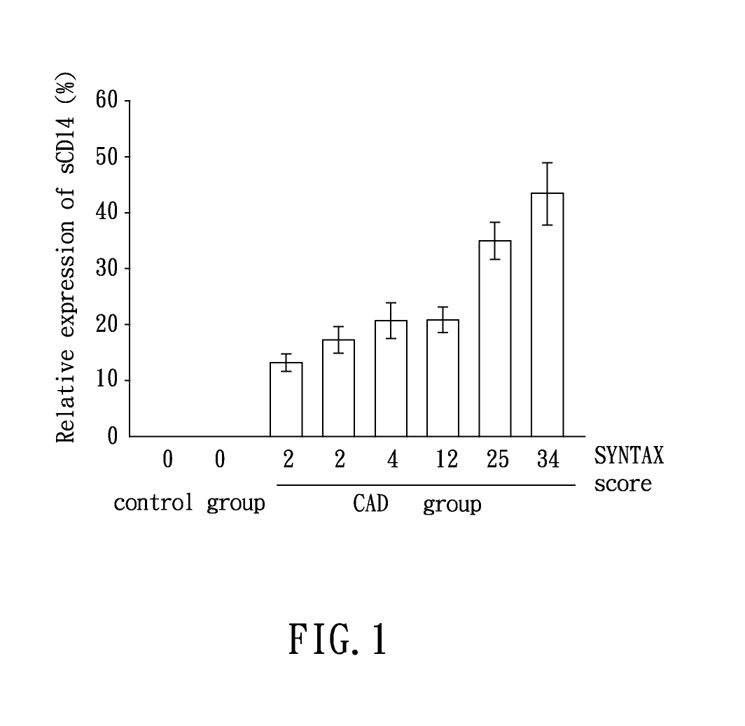

As shown in FIG. 1, the level of sCD14 is increased in the CAD group when compared to the control group. Moreover, a positive correlation between the level of sCD14 and the SYNTAX score is shown (p for trend <0.001). These results indicate that the level of sCD14 in urine samples can be a biomarker to detect CAD.

### Example 2

### Analyses of the Level of sCD14 Between the Control and CAD Groups in Serum or Urine

In order to determine the association between the level of sCD14 and the severity of CAD, the level of sCD14 in serum or urine was assessed using enzyme linked immunosorbent assay (ELISA).

### Method:

Serum and urine samples were collected from 73 CAD patients, either with single- or multi-vessel disease, and 35 healthy subjects. The serum and urine samples were collected using the procedures mentioned in the aforesaid <Sample collection> section.

sCD14 levels were determined using human sCD14 enzyme linked immunosorbent assay kit (catalog number: HK320, Hycult Biotechnology, Uden, the Netherlands) based on the manufacturer's instructions.

A receiver operating characteristic (ROC) curve was obtained by the analyses of sCD14 levels from the studied subjects using SPSS 16.00 software (SPSS, Chicago, Ill.). An area under the ROC curve (AUC) for urinary sCD14 was calculated and used as an index to determine whether urinary sCD14 had a good diagnostic ability to distinguish patients from healthy subjects.

### Results:

The average levels of sCD14 from both serum and urine samples obtained from the control and CAD groups are shown in Table 2. The serum sCD14 levels do not differ between the control and CAD groups. However, compared to the control group, the level of urinary sCD14 is significantly increased in the CAD group, with patients either having single-vessel or multi-vessel disease. In addition, a trend of increased level of urinary sCD14 was observed in multi-vessel disease patients when compared to single-vessel disease patients.

TABLE 2

| | Group | | |
|---|---|---|---|
| | | CAD | |
| | | Single-vessel | Multi-vessel |
| | Control | disease | disease |
| | (n = 35) | (n = 20) | (n = 53) |
| Concen- | 132.81 ± 37.51 | 122.32 ± 39.67 | 132.92 ± 36.74 |
| tration | | | |
| of serum | | | |
| sCD14 | | | |
| (μg/mL) | | | |
| Concen- | 2.08 ± 2.02 | 9.55 ± 12.36* | 11.1 ± 10.92**^{#} |
| tration | | | |
| of urinary | | | |
| SCD14 | | | |
| (μg/mL) | | | |

*Single-vessel disease CAD patients vs. controls, p < 0.001
**Multi-vessel disease CAD patients vs. controls, p < 0.001
^{#}Single-vessel disease CAD patients vs. Multi-vessel disease CAD patients p = 0.605

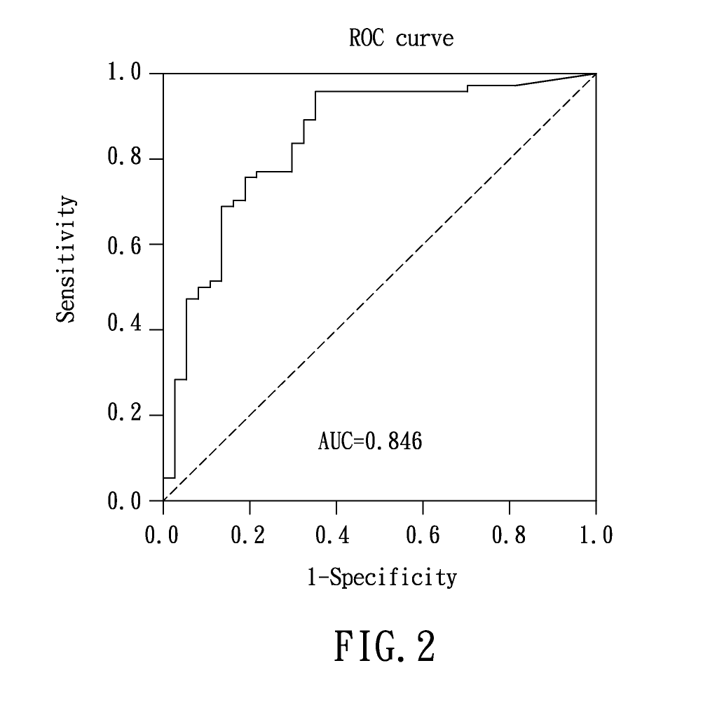

Accuracy of a diagnostic method is best described as its receiver operating characteristic (see Zweig M. H. Clin. Chem. 39, 561-577, 1993). A graph of an ROC curve is a plot of all of the sensitivity/specificity pairs resulting from continuously varying the decision threshold over the entire range of data observed. On the Y-axis, sensitivity is calculated solely from the CAD group. On the X-axis, sensitivity is calculated entirely from the control group. Each point on the ROC curve represents a sensitivity/specificity pair corresponding to a particular decision threshold. The ROC curve shown in FIG. 2 suggests that the standard value for CAD is 3.51 μg/mL, with the sensitivity and specificity being 0.838 and 0.703 respectively. These results suggest that the level of urinary sCD14 that is equal to or greater than 3.51 μg/mL is indicative of a human subject having CAD.

The area under the ROC curve (AUC) is frequently used as an evaluation of the usefulness among different diagnostic methods (see Zweig M. H. Clin. Chem. 39,561-577, 1993). Interpretations of AUC value include: outstanding (AUC>0.9), excellent (AUC=0.8˜0.9), acceptable (AUC=0.7-0.8), poor (AUC=0.6˜0.7), and no discrimination (AUC=0.5). As shown in FIG. 2, the AUC is 0.846, suggesting an excellent discrimination between healthy subjects and CAD patients.

To sum up, urinary sCD14 is a superior biomarker for CAD when compared to serum sCD14, and provides better sensitivity and specificity. Thus, the inventors believe that urinary sCD14 is a more superior biomarker for CAD than serum sCD14.

All patents and literature references cited in the present specification as well as the references described therein, are hereby incorporated by reference in their entirety. In case of conflict, the present description, including definitions, will prevail.

While this invention has been described in connection with specific embodiments thereof, it will be understood that it is capable of further modifications and this application is intended to cover any variations, uses, or adaptations of the invention following, in general, the principles of the invention and including such departures from the present customary practice within the art to which the invention pertains and as may be applied to the essential features hereinbefore set forth, and as follows in the scope of the appended claims.

What is claimed is:

1. A method for the detection or preliminary screening of coronary artery disease, comprising:
obtaining a urine sample from a human subject suspected of having coronary artery disease;
detecting a level of sCD14 in the urine sample from the human subject suspected of having coronary artery disease; and
comparing the detected level of sCD14 in the urine sample with a predetermined standard;
wherein the level of sCD14 in the urine sample from the human subject suspected of having coronary artery disease greater than the predetermined standard is indicative of the presence of coronary artery disease.

2. The method according to claim 1, wherein the level of sCD14 is determined using an antibody-based binding moiety which specifically binds to sCD14.

3. The method according to claim 2, wherein the level of sCD14 is determined by multiplex immunoassay, enzyme linked immunosorbent assay, radioimmunoassay, immunoradiometric assay, fluorescent immunoassay, chemiluminescent immunoassay or immunonephelometry.

4. The method according to claim 2, wherein the antibody-based binding moiety is an antibody.

5. The method according to claim 2, wherein the binding of the antibody is labeled with a detectable label.

6. The method according to claim 5, wherein the detectable label is selected from the group consisting of a radioactive label, a hapten label, a fluorescent label, a chemiluminescent label, an enzymatic label and an epitope tag.

7. The method according to claim 1, wherein the level of urinary sCD14 is determined by enzyme linked immunosorbent assay, and the predetermined standard for sCD14 is 3.51 μg/mL.

8. A method for the monitoring of coronary artery disease, comprising:
periodically obtaining a urine sample from a human subject suspected of having coronary artery disease;
detecting a level of sCD14 in the urine sample from the human subject suspected of having coronary artery disease; and
comparing the detected level of sCD14 in the urine sample with a predetermined standard;
wherein the level of sCD14 in the urine sample from the human subject suspected of having coronary artery disease greater than the predetermined standard is indicative of the presence of coronary artery disease.

9. The method according to claim 8, wherein the level of sCD14 is determined using an antibody-based binding moiety which specifically binds to sCD14.

10. The method according to claim 9, wherein the level of sCD14 is determined by multiplex immunoassay, enzyme linked immunosorbent assay, radioimmunoassay, immunoradiometric assay, fluorescent immunoassay, chemiluminescent immunoassay or immunonephelometry.

11. The method according to claim 9, wherein the antibody-based binding moiety is an antibody.

12. The method according to claim 9, wherein the binding of the antibody is labeled with a detectable label.

13. The method according to claim 12, wherein the detectable label is selected from the group consisting of a radioactive label, a hapten label, a fluorescent label, a chemiluminescent label, an enzymatic label and an epitope tag.

14. The method according to claim 8, wherein the level of urinary sCD14 is determined by enzyme linked immunosorbent assay, and the predetermined standard for sCD14 is 3.51 μg/mL.
